(19) United States
(12) Patent Application Publication
(10) Pub. No.: US 2016/0310754 A1
(43) Pub. Date: Oct. 27, 2016

(54) SYSTEMS AND METHODS FOR CONTROLLING ANIMAL BEHAVIOR VIA OPTOGENETICS

(71) Applicant: Elwha LLC, Bellevue, WA (US)

(72) Inventors: Mahalaxmi G. Bangera, Renton, WA (US); Roderick A. Hyde, Redmond, WA (US)

(73) Assignee: ELWHA LLC, Bellevue, WA (US)

(21) Appl. No.: 14/694,727

(22) Filed: Apr. 23, 2015

(51) Int. Cl. A61N 5/06; C12N 15/85; A01K 67/033; C07K 14/705

(52) U.S. Cl. CPC A61N 5/062; C07K 14/705; C12N 15/8509; A01K 67/0339; A61N 5/0622; A61N 5/0618; A01K 2227/706; A01K 2217/052; A61N 2005/0626; A61N 2005/0663

(57) ABSTRACT

A method includes applying, by an optical stimulation device, an optical stimulus to an olfactory organ of an animal having a genetically engineered olfactory sensory protein to produce a desired neural response. The genetically engineered olfactory sensory protein has been genetically modified to incorporate a light-responsive element.

### BACKGROUND

Animals, such as insects, use olfaction to sense their surroundings and to guide important activities, such as feeding, mating, oviposition, etc. For example, insects sense odors through the activation of various odorant receptors (“ORs”), which are a certain type of G protein-coupled receptor (“GPCR”). A functional OR complex includes both a conventional odorant-binding OR and a nonconventional coreceptor (“Orco”). An OR is responsible for binding to a ligand/odorant and, in combination with an Orco, forming an odorant-gated ion channel to enable chemosensation. Once a ligand/odorant is bound, a cascade of events is initiated that leads to nervous activity.

ORs of insects have been identified and characterized. For example, 78 conventional ORs have been identified and characterized in Anopheles gambiae (An. gambiae), commonly known as a mosquito. A single conventional OR is expressed in every olfactory receptor neuron (ORN) in conjunction with an Orco. The An. gambiae Orco is common across all of its ORs. Thus, activating the An. gambiae Orco also activates all of its associated ORs. In addition, Orcos are highly-conserved across insect taxa.

### SUMMARY

One embodiment relates to a method including applying, by an optical stimulation device, an optical stimulus to an olfactory organ of an animal having a genetically engineered olfactory sensory protein to produce a desired neural response. The genetically engineered olfactory sensory protein has been genetically modified to incorporate a light-responsive element.

Another embodiment relates to an optical stimulation device. The optical stimulation device includes a light source and a processing circuit operably coupled to the light source. The processing circuit includes an optical stimulation module configured to receive optical sensitivity data regarding optical sensitivity of a genetically engineered olfactory sensory protein of an animal. The processing circuit is also configured to receive a response command regarding a desired behavioral response of the animal. The processing circuit is further configured to determine, based on the optical sensitivity data and the response command, an optical stimulus. The optical stimulus, when provided to the animal, is configured to cause the animal to exhibit the desired behavioral response. Further yet, the processing circuit is configured to transmit, to the light source, a control signal to cause the light source to provide the optical stimulus to the animal.

Another embodiment relates to a method, including receiving, by a processor, optical sensitivity data regarding optical sensitivity of a genetically engineered olfactory sensory protein of an animal. The method also includes receiving, by the processor, a response command regarding a desired behavioral response of the animal. The method further includes determining, by the processor, based on the optical sensitivity data and the response command, an optical stimulus. The optical stimulus, when provided to the animal, is configured to cause the animal to exhibit the desired behavioral response. Further yet, the method includes transmitting, by the processor, to the light source, a control signal to cause the light source to provide the optical stimulus to the animal.

Another embodiment relates to a method, including identifying a target olfactory sensory protein of an animal. The method also includes genetically engineering the target olfactory sensory protein to incorporate a light-responsive element. The light-responsive element is configured to produce a desired neural response when an optical stimulus is applied to an olfactory organ of the animal that includes the light-responsive element.

The foregoing summary is illustrative only and is not intended to be in any way limiting. In addition to the illustrative aspects, embodiments, and features described above, further aspects, and embodiments, and features will become apparent by reference to the drawings and the following detailed description.

### BRIEF DESCRIPTION OF THE DRAWINGS

FIG. 1A is a diagram of an insect of the species An. gambiae, according to one embodiment.

FIG. 1B is a detail view of antennae of the insect of FIG. 1A, including sensillum protruding from the antennae.

FIG. 1C is a detail view of the sensillum and dendrite of FIG. 1B.

FIG. 2 is a flow diagram of a method of introducing a light-responsive element into a target olfactory sensory protein of an animal to produce an engineered target olfactory sensory protein, according to an embodiment.

FIG. 3 is an illustration of a system including an optical stimulation device, according to an embodiment.

FIG. 4 is a block diagram of the optical stimulation device of FIG. 3, according to an embodiment.

FIG. 5 is a flow diagram of a method of operating the optical stimulation device of FIGS. 3 and 4, according to an embodiment.

### DETAILED DESCRIPTION

In the following detailed description, reference is made to the accompanying drawings, which form a part thereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Optogenetics involves enabling specific physiological processes to be controlled by light. Optogenetics involves the discovery and insertion into cells of genes that confer light responsiveness. An introductory discussion of optogenetics is available as “Controlling the Brain with Light,” Karl Deisserot, Scientific American, November 2010, p 49. A more detailed review is provided by “The Development and Application of Optogenetics,” Lief Fenno, Ofer Yizhar, and Karl Deisseroth, Annual Review of Neuroscience Vol. 34: 389-412. A key feature of optogenetics is millisecond-scale temporal precision, which allows neural activity to be controlled at the speed at which neurons operate. For example, even a shift of a few milliseconds in the timing of a neuron's firing can completely reverse the effect of its signal on the rest of the nervous system.

Certain proteins present in microbes, such as G-Protein Coupled Receptor (GPCR) opsins, have light-activated ion channels that regulate the flow of positively charged ions across the channels in response to exposure to certain types of light. The genes of these proteins may be spliced into the genes of target cells, such as neurons, to enable precise (e.g., millisecond scale) control over the cells' functionality. The GPCR opsins that may be used in this technique include, for example, rhodopsins, channelrhodopsins (e.g., ChR2, ChR1, VChR1, and SFOs), halorhodopsins, archaerhodopsins, bacteriorhodopsins, etc. Genetically engineered GPCR-opsin combinations have been demonstrated to control cellular responses by at least three different pathways; the modulation of ion-channel conductances, the release of intracellular Ca2+ stores, and via intracellular second messengers such as cAMP. Discussions of such control are provided by “Light and Drug-activated G-Protein-Coupled Receptors to Control Intracellular Signalling,” Masseck, Rubelowski, Spoida, Herlitze, Experimental Physiology, Vol 96, Issue 1, p 51 (2011), by “Temporally Precise in-vivo Control of Intracellular Signalling,” Airan, Thompson, Fenno, Bernstein, Deisseroth, Nature, Vol 458, p 1025 (2009), by “Molecular and Cellular Approaches for Diversifying and Extending Optogenetics,” Gradinaru, et al, Cell, Vol 141, p 1 (2010), and by “The Microbial Opsin Family of Optogenetic Tools,” Zhang, et al, Cell, Vol 147, p 1446 (2011). Using techniques reviewed or referenced in these documents, optical control of cells has been extended to longer wavelengths, to faster kinetics, to increased light sensitivity, to bi-stable (i.e., latched-in) activation or deactivation, to non-GPCR molecules, etc.

One significant challenge associated with optogenetics is accessing the target neurons so that they may be controlled via light. For many animals, such as mice and humans, the neurons are located in the brain, which is protected by the animals' skull. Accordingly, the target neurons are typically accessed via invasive techniques in which an optical fiber is inserted through the animal's skull and into the brain. Such techniques are complicated, time consuming, and may result in undesirable side effects resulting from displaced brain tissue.

In contrast, many insects, such as mosquitoes (e.g., An. gambiae) and honey bees (e.g., Apis mellifera), for example, have olfactory sensory proteins including GPCR olfactory sensory elements (e.g., OR and Orco) located in their antennae, which can be genetically engineered so that they can be excited via light using optogenetics just as they currently can be excited via odors, without requiring invasive techniques. A general review of olfactory receptors is provided by “Olfactory Receptors: G Protein-Coupled Receptors and Beyond,” Spehr, Munger, Journal of Neurochemistry, Vol 109, p 1570 (2009). A discussion of chemically controlling insect olfactory receptors as well as the ORCO coreceptor is provided by “Functional Agonism of Insect Odorant Receptor Ion Channels,” Jones, Pask, Rinker, Zwiebel, Proceedings of the National Academy of Sciences, Vol 108, p 8821 (2011). In addition, because insects rely heavily on olfaction to sense their surroundings and to guide important activities such as feeding, mating, ovipositing, etc., the ability to optically control insects' olfactory sensations and neural pathways provides the ability to control much of the insects' behavior, including their movement (e.g., locomotion and orientation).

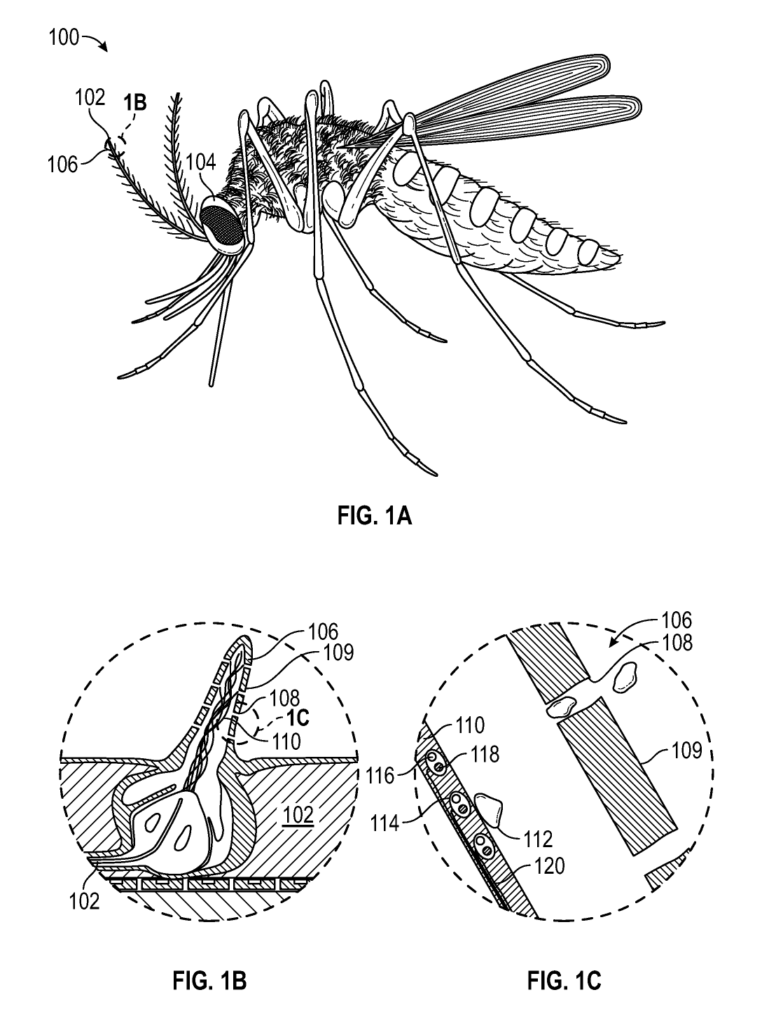

FIG. 1A is a diagram of insect 100 of the species An. gambiae. Insect 100 includes antennae 102 extending from head 104. A plurality of sensilla 106 protrude from each antenna 102.

FIG. 1B is a detail view of antennae 102 of insect 100 of FIG. 1A, including sensillum 106 protruding from antennae 102. Odorants enter sensillum 106 through tiny pores 108 in sensillum wall 109. Once inside sensillum 106, the odorants interact with olfactory sensory proteins located on dendrites 110 within sensillum 106.

FIG. 1C is a detail view of sensillum 106 and dendrite 110 of FIG. 1B. As shown in FIG. 1C, Upon entering sensillum 106, odorant 112 binds with one or more olfactory sensory proteins 114 located on dendrite 110. Each olfactory sensory protein 114 may include, for example, conventional OR 116 and common Orco 118. Upon binding, OR 116, in combination with Orco 118, forms an odorant-gated ion channel. A cascade of events results in a neural response, which is transmitted to the brain via axons 120, where the response is perceived as a particular odorant. The perception of particular odorants may cause a particular behavioral response of insect 100. For example, perception of a first odor (e.g., an attractant) may cause locomotion of animal 100 towards a source of the first odor, and perception of second odor (e.g., a repellent) may cause locomotion of animal 100 away from a source of the second odor.

By understanding the underlying physiology and neural mechanisms of animals' olfaction, various natural and synthetic devices may be utilized to control the animals' behavior via olfaction. For example, various agonists (e.g., insecticides and insect repellants) have been identified and developed to manipulate insects' behavior through olfactory stimulation. Certain agonists target specific ORs, while other agonists target a specific Orco, which can control all of an insect's ORs. However, such agonists may kill or harm other creatures in addition to those they are intended to kill, and may also harm the environment.

The present disclosure relates to introducing light-responsive elements into target olfactory sensory proteins (e.g., ORs or Orcos) of animals (e.g., insects), to produce an genetically engineered olfactory sensory protein, such that applying an optical stimulus to the genetically engineered olfactory sensory proteins causes the genetically engineered olfactory sensory protein to produce a desired neural response. In some embodiments, the introduction of the light-responsive elements is performed genetically, by introducing nucleic acid encoding the light-responsive elements into nucleic acid encoding an olfactory sensory protein. This introduction may be purely additive, or may involve the removal or replacement of a portion of the nucleic acid encoding the original olfactory sensory protein.

As used herein, the term “light responsive element” refers to a functional opsin protein, a naturally or artificially occurring functional variant thereof, or a functional fragments thereof. As used herein, the term “functional” means responsive to light. As used herein, the term “functional fragment” refers to a less than a full-length opsin protein or variant (e.g., an opsin polypeptide fragment) that is responsive to light. In some embodiments, a light responsive element includes an opsin protein, a naturally or artificially occurring functional variant thereof, or a functional fragment thereof in combination with one or more chromophores, or other natural or artificial light-responsive compounds.

In some embodiments, the functional opsin protein, variant or fragment thereof comprises or is derived from, a Type 1, or prokaryotic, opsin. In some embodiments, the functional opsin protein, variant or fragment comprises or is derived from a Type 2, or eukaryotic, opsin. In some embodiments, the opsin protein, variant or functional fragment comprises, or is derived from both a Type 1 and Type 2 opsin and is responsive to light.

In some embodiments, the light-responsive element may itself constitute a modification of a naturally occurring opsin protein. It may have been genetically engineered or genetically evolved so as to improve efficacy (e.g., optical sensitivity, compatibility with a target olfactory sensory protein into which it will be introduced, compatibility with an animal in which it will be introduced, etc.). The desired neural response may be configured to cause a desired behavioral response of the animal. In an embodiment, an optical stimulation device is configured to transmit an optical stimulus (e.g., a light beam) to the animal from a light source. The optical stimulus may have certain properties (e.g., wavelength and intensity), such that the optical stimulus is configured to control a behavior (e.g., locomotion) of the animal.

According to an embodiment, applying an optical stimulus (e.g., a light beam) to a genetically engineered olfactory sensory protein that includes a light-responsive element may cause a desired neural response that is perceived by the animal as a particular odor. The perception of the particular odor may cause a desired behavioral response of the animal. For example, the particular behavior may include attraction to or repulsion from a source of the stimulus.

Controlling animals' behavior via optogenetics provides many advantages over controlling animals' behavior via chemical stimuli (e.g., repellents) due to, for example, the ability to precisely control optical stimuli. For example, optical stimuli may be spatially and temporally controlled with much greater precision than chemical stimuli. In addition, chemical stimuli may cause significant collateral damage to the environment and other organisms proximate its application, whereas optical stimuli are generally benign to non-modified organisms.

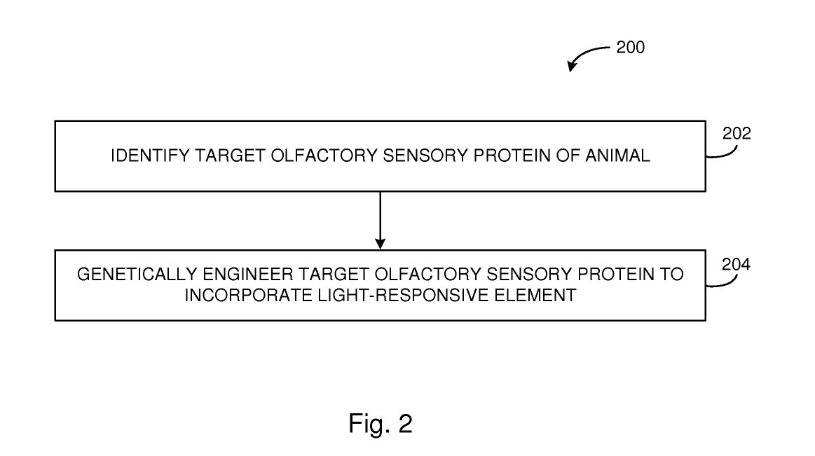

FIG. 2 is a flow diagram of a method 200 of introducing a light-responsive element into a target olfactory sensory protein (e.g., which may include genes that encode ORs or Orcos) of an animal to provide an engineered target olfactory sensory protein, such that applying an optical stimulus to the engineered target olfactory sensory protein causes the target olfactory sensory protein to produce a desired neural response. Method 200 is described below as applied to an insect and, more specifically, as applied to An. gambiae. However, method 200 may be similarly applied to other insects, as well as other animals.

At 202, a target olfactory sensory protein of the animal is identified. According to various embodiments, the target olfactory sensory protein may include an OR or an Orco. In some embodiments, the target olfactory sensory protein includes a combination of particular ORs. As mentioned above, An. gambiae has 78 known ORs. An. gambiae is capable of sensing certain odors based on the particular OR or the particular combination of certain ORs that are activated. Activation of certain ORs or combinations thereof may determine particular odorant identities, which in turn may indicate whether the odorant is perceived as an attractant or a repellant. Accordingly, the particular ORs or combinations thereof may be selected based on the desired behavioral response (e.g., attraction or repellent) caused by activating those particular ORs or combinations thereof.

At 204, the target olfactory sensory protein is genetically engineered to include a light-responsive element. The light-responsive element is configured in the genetically engineered olfactory sensory protein such that applying an optical stimulus to the genetically engineered olfactory sensory protein causes the light-responsive element to produce a desired neural response. In effect, applying the optical stimulus to the target olfactory sensory protein operates to simulate a particular perceived odorant identity. The particular perceived odorant identity can determine whether the animal perceives the simulated odorant as an attractant (e.g., a food source) or a repellant (e.g., a predator). Accordingly, the particular perceived odorant identity can dictate the animal's behavior, such as orientation and locomotion.

According to various embodiments, nucleic acid encoding the light-responsive elements is introduced into the target olfactory sensory protein in various ways, such as through non-viral transfection (e.g., via electroporation or by use of a gene gun) or through viral transfection (e.g., via a bacteriophage), which in some implementations may employ the use of a gene drive.

According to various embodiments, insects are genetically modified in a controlled environment (e.g., a laboratory), where the genetically modified (transgenic) insects may be expanded by selective breeding, and the offspring may subsequently be released into the wild. In certain implementations, the insects are genetically modified to include other genetic enhancements in addition to those encoding the light-responsive elements to provide selective advantages to the transgenic insects over wild-type insects. For example, such genetic enhancements may provide advantages relating to health, fitness, sensory acuity, reproductive ability, etc., to further the proliferation of the genetically engineered olfactory sensory protein in the insect population.

In some embodiments, gene drives are utilized to genetically modify the animals. Gene drives are used to stimulate biased inheritance of particular genes to alter entire populations of organisms. For example, a gene-editing technique called CRISPR/Cas9 may be used to build endonuclease gene drives; this is discussed in “Concerning RNA-guided gene drives for the alteration of wild populations,” Kevin M Esvelt, Andrea L Smidler, Flaminia Catteruccia, George M Church, eLife 2014; 10.7554/eLife.03401 (DOI: http://dx.doi.org/10.7554/eLife.03401). Clustered regularly interspaced short palindromic repeats (CRISPRs) are DNA loci containing short repetitions of base sequences. Each repetition is followed by short segments of “spacer DNA” from previous exposures to a virus. CRISPRs are found in approximately 40% of sequenced bacteria genomes and 90% of sequenced archaea. Cas9 is a non-repetitive enzyme that can be directed to cut almost any DNA sequence by simply expressing a “guide RNA” containing that same sequence. Editing genomes with CRISPR involves expressing an RNA-guided Cas9 endonuclease along with guide RNAs directing Cas9 to cut particular sequences to be edited. When Cas9 cuts the target sequences, the target cell is induced to repair the damage by replacing the original sequence with a replacement gene (or nucleic acid sequence). A gene drive can be implemented using CRISPR by including DNA encoding Cas9 and the target guide RNA along with that for the desired genetic modification. In the instant case, the target cell may express the target olfactory sensory protein and the replacement gene (or nucleic acid sequence) may encode a light-responsive element.

In some embodiments, different types (e.g., species) of animals are genetically modified with different light-responsive elements. For example, in an embodiment, a first light-responsive element may be introduced into a first target olfactory sensory protein of a first animal to form a first genetically engineered olfactory sensory protein, and a second light-responsive element may be introduced into a second target olfactory sensory protein of a second animal to form a second genetically engineered olfactory sensory protein. The first genetically engineered olfactory sensory protein, may produce a first desired neural response when provided with a first optical stimulus, and the second genetically engineered olfactory sensory protein, may produce a second desired neural response when provided with a second optical stimulus. The first desired neural response may be configured to cause a first desired behavioral response of the first animal, and the second desired neural response may be configured to cause a second desired behavioral response of the second animal. Each of the first and second genetically engineered olfactory sensory proteins, the first and second desired neural responses, the first and second desired behavioral responses, and the first and second optical stimuli may be the same or different.

In an embodiment, the first and second animals are of different species. For example, the first animal may be An. gambiae and the second animal may be Apis mellifera. The first and second genetically engineered olfactory sensory proteins may respond differently to different optical stimuli. For example, the first optical stimulus that produces the first desired neural response may have a first wavelength and a first intensity, whereas the second optical stimulus that produces a desired neural response may have a second wavelength and a second intensity. The first optical stimulus may not cause a neural response in the second animal, and vice versa. Accordingly, in some embodiments, the behavior of different species of animals can be independently and simultaneously controlled via optical stimulation control.

In some embodiments, a single animal is genetically modified by introducing light-responsive sequences into multiple target olfactory sensory proteins (e.g., ORs and/or Orcos) forming multiple genetically engineered olfactory sensory proteins. The multiple genetically engineered olfactory sensory proteins may be the same or different. Because animals perceive certain odors based on the activation of particular olfactory sensory proteins or combinations thereof, such embodiments facilitate simulation of multiple different odors. Because animals' behavior may be controlled by olfaction, the ability to simulate multiple perceived odors provides a greater degree of control over the animals' behavior.

For example, in an embodiment, an animal is genetically modified such that providing a first optical stimulus operates to simulate a strong repellent, and providing a second optical stimulus operates to simulate a mild attractant. For example, the first optical stimulus may have a wavelength corresponding to green light (e.g., 532 nm wavelength) and the second optical stimulus may have a wavelength corresponding to blue light (e.g., 480 nm wavelength). For example, the first optical stimulus may latch-in a first response (e.g., so that the first response persists even after termination of the first optical stimulus) and the second optical stimulus may only cause the second response while the second optical stimulus persists. Further, the first and second optical stimuli may be provided at different intensities. By independently varying the first and/or second optical stimuli over time, the behavioral response of the animal may be controlled with greater precision than if only one optical stimulus was used.

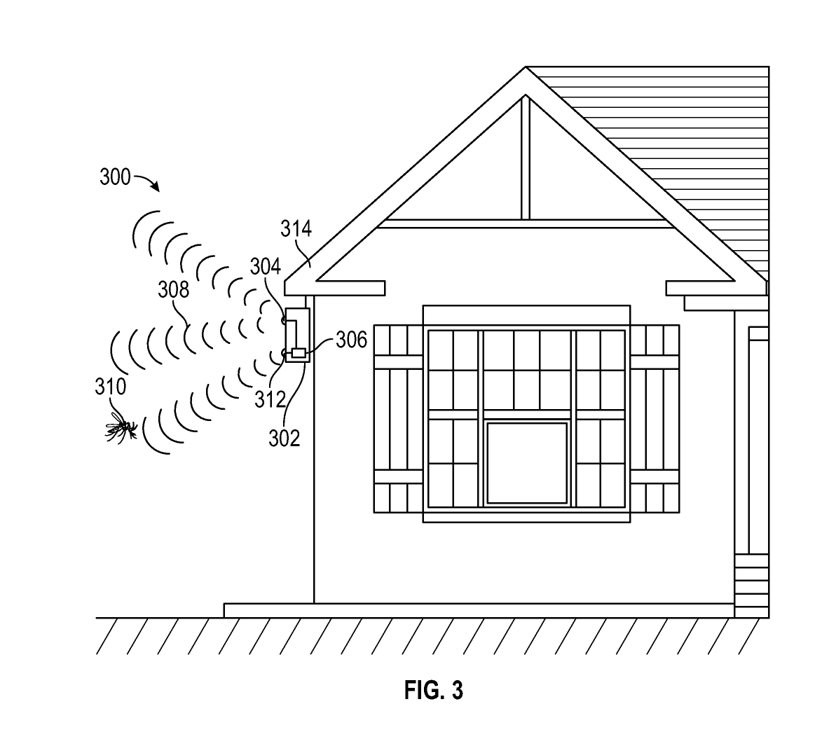

Referring to FIG. 3, system 300 including optical stimulation device 302 is illustrated, according to an embodiment. According to an embodiment, optical stimulation device 302 includes light source 304 operably coupled to processing circuit 306. Processing circuit 306 is configured to provide a control signal to light source 304 to cause light source 304 to provide optical stimulus 308 (e.g., light beams) to insect 310. Optical stimulus 308 may be configured to control a desired behavioral response of insect 310, which has been genetically modified. In an embodiment, the genetic modification of insect 310 includes nucleic acid encoding a light-responsive element introduced into a target olfactory sensory protein (e.g., thereby encoding the target olfactory sensory protein) of insect 310, such that upon expression of the genetically engineered target olfactory sensory protein, applying optical stimulus 308 to the genetically engineered target olfactory sensory protein causes the genetically engineered target olfactory sensory protein to produce a desired neural response. The desired neural response may be configured to cause a desired behavioral response of insect 310. Although system 300 of FIG. 3 is illustrated with respect to insect 310, system 300 is similarly usable with other types of animals. According to various embodiments, system 300 is primarily utilized in low-light conditions (e.g., at nighttime) to maximize the effect of optical stimulus 308.

Optical stimulation device 302 may also include animal position sensor 314 operably coupled to processing circuit 306. In an embodiment, animal position sensor 314 is configured to monitor the actual behavior of insect 310. Animal position sensor 312 may be image-based (e.g., camera-based), radar-based, or acoustic-based. Animal position sensor 314 is configured to communicate a measurement signal to processing circuit 306, where the measurement signal may be analyzed. Processing circuit 306 may identify animals based on optical image recognition techniques, as well as based on certain types of motion that are characteristic to particular animals (e.g., insect wing beat frequency). In an embodiment, processing circuit 306 is configured to compare the desired behavioral response of insect 310 with the actual behavior of insect 310, and to adjust the control signal transmitted to light source 304 based on the comparison to provide closed-loop insect 310 behavior control.

As illustrated in FIG. 3, optical stimulation device 302 may be mounted to structure 314 (e.g., a building) to improve the coverage of optical stimulus 308. In other embodiments, optical stimulation device 302 is a handheld device. In still other embodiments, optical stimulation device 302 is mobile. For example, in an embodiment, optical stimulation device 302 is mounted to a ground-based or air-based vehicle (e.g., an unmanned aerial vehicle).

As used herein, the terms “optical stimulus,” “light,” or “light beam” may refer to electromagnetic radiation having a wavelength and an intensity, and may include visible light, infrared light, and ultraviolet light. According to an embodiment, optical stimulus 308 may be delivered according to various parameters. In some implementations, various parameters of optical stimulus 308, such as wavelength and intensity, are varied over time. For example, optical stimulus 308 may be delivered continuously at a single wavelength and a single intensity, at a single wavelength at varying intensities, etc. Put another way, optical stimulus 308 may be delivered continuously or may be delivered as pulses, bursts, and/or other patterns that vary over time. Optical stimulus 308 may be selected based on knowledge of the optical responsiveness of an identified genetically engineered olfactory sensory protein. Optical stimulus 308 may be selected to latch-in an optical response or may be selected to induce the response only while optical stimulus 308 is present.

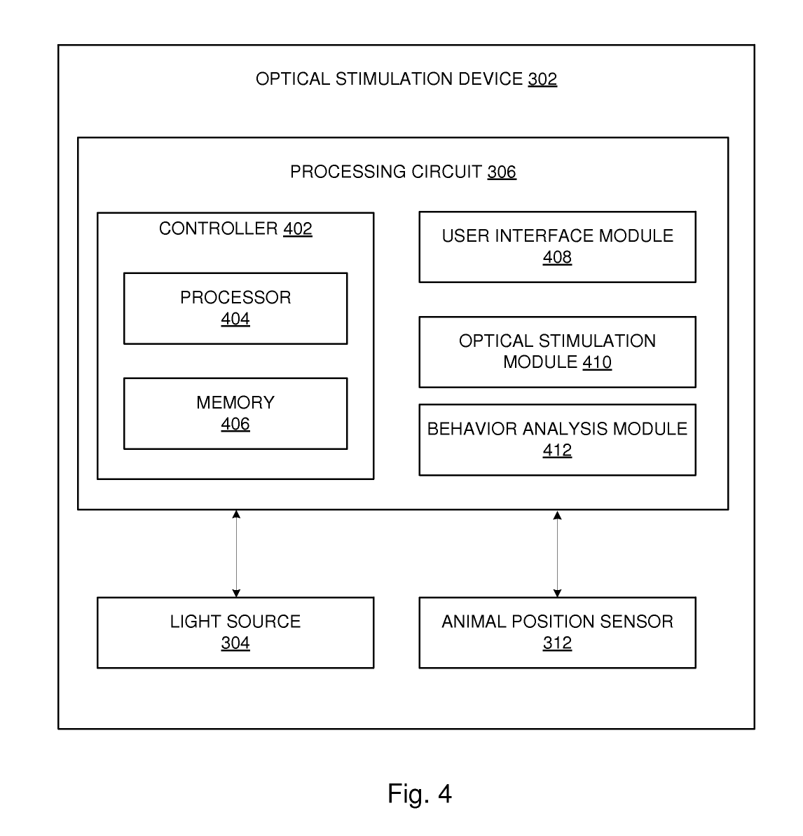

Referring to FIG. 4, a block diagram of optical stimulation device 302 of FIG. 3 is illustrated, according to an embodiment. As illustrated in FIG. 4, optical stimulation device 302 includes processing circuit 306, light source 304, and animal position sensor 312. Processing circuit 306 includes controller 402, which controls the various modules of processing circuit 300. Controller 402 includes processor 404 and memory 406. Processor 404 may be implemented as a general-purpose processor, an application specific integrated circuit (ASIC), one or more field programmable gate arrays (FPGAs), a digital-signal-processor (DSP), a group of processing components, or other suitable electronic processing components. Memory 406 is one or more devices (e.g., RAM, ROM, Flash Memory, hard disk storage, etc.) for storing data and/or computer code for facilitating the various processes described herein. Memory 406 may be or include non-transient volatile memory or non-volatile memory. Memory 406 may include database components, object code components, script components, or any other type of information structure for supporting the various activities and information structures described herein. Memory 406 may be communicably connected to processor 404 and provide computer code or instructions to processor 404 for executing the processes described herein.

Controller 402 is in communication with user interface module 408, optical stimulation module 410, and behavior analysis module 412, which control various aspects of the operation of optical stimulation device 302.

User interface module 408 is configured to provide various outputs to and to receive various inputs from a user. For example, in some embodiments, user interface module 408 is configured to display information on a screen (e.g., an LCD screen) and receive inputs from an input device (e.g., a touchscreen or buttons) integrated with optical stimulation device 302. In other embodiments, user interface module 408 is configured to display information on and receive inputs from a remote computing device (e.g., a desktop or laptop computer, a tablet, a smartphone, a mobile device, etc.) separate from optical stimulation device 302. In some embodiments, user interface module 408 includes audio components such as a microphone to accept verbal input and/or a speaker to provide audible output.

Optical stimulation module 410 receives inputs from user interface module 408 and determines the properties (e.g., wavelength and intensity, which may be time-variable) of the optical stimulus to be provided by light source 304. Controller 402, in communication with user interface module 408 and optical stimulation module 410, controls the operation of light source 304 based on the user inputs from optical stimulation module 410 and based on further control parameters determined by optical stimulation module 410.

According to various embodiments, user interface module 408 can receive inputs relating to various aspects of operation of optical stimulation device 302, including inputs defining the target animal (e.g., insect 310) and a desired behavior of the target animal. For example, user interface module 408 may receive an input to target a certain species, such as An. gambiae. As discussed above, certain genetically engineered olfactory sensory proteins respond to different optical stimuli. For example, some may respond to light having a first wavelength (e.g., green light, 532 nm wavelength) and others may respond to light having a second wavelength different than the first wavelength (e.g., blue light, 480 nm wavelength). In some implementations, multiple species may be genetically modified with different genetically engineered olfactory sensory proteins, such that optical stimulation at a first wavelength modifies the behavior of a first species and optical stimulation at a second wavelength modifies the behavior of a second species.

User interface module 408 can also receive inputs relating to the desired behavior of the target animal. For example, the desired behavior may be to repel the target animal, to attract the target animal, to guide the target animal to a particular target, or to confuse the target animal. For example, stimulation of a particular OR at a particular wavelength and intensity may cause the target animal to perceive the stimulus as an attractant odor or as a repellent odor. In some implementations, an optical stimulus including a constant light beam at a particular wavelength and intensity may cause a constant repellent or attractive effect. In some implementations, an optical stimulus including a light beam with a time-variable wavelength and/or frequency is provided to control the movement of the target animal.

In some embodiments, user interface module 408 may receive an input from a user to terminally confuse the target animal. Based on the inputs received via user interface module 408, optical stimulation module 410 may terminally confuse the target animal in various ways. For example, optical stimulation module 410 may vary at least one of the wavelength and intensity of the optical stimulus over time. In an embodiment, the intensity is cycled between an ON level and an OFF level. In another embodiment in which the target animal has been genetically modified with different light-responsive elements, optical stimulation module 410 may cycle the optical stimulus between different frequencies, such that the target animal perceives an attractant and then a repellant in a cyclic manner. In other embodiments in which the target olfactory sensory protein includes an Orco, activation of the Orco activates all of the insect's associated ORs. This may also terminally confuse the target animal.

In some embodiments, the target animal may be of the genus Apis (e.g., Apis mellifera or Apis cerana indica), commonly referred to as a honeybee. In certain embodiments, the target animal is genetically modified to include nucleic acid encoding a light-responsive element in one or more of its olfactory sensory proteins, such that optical stimulation of the genetically engineered olfactory sensory proteins operates to simulate the scent of nectar. In these embodiments, the animals may be attracted to a particular grouping of plants (e.g., a particular field), such that the animals pollinate the plants in that grouping.

Behavior analysis module 412 is configured to receive various inputs from animal position sensor 312 to identify and monitor the actual behavior of the target animals. Animal position sensor 312 may be image-based (e.g., camera-based), radar-based, or acoustic-based. For example, behavior analysis module 412 may identify insects based on optical image recognition techniques, as well as based on certain types of motion that are characteristic to particular animals (e.g., insect wing beat frequency). Optical stimulation module 410 may control the operation of the light source 304 based on the actual behavior determined by the behavior analysis module 412. In an embodiment, processing circuit 306 is configured to compare the desired behavioral response of the target animal with the actual behavior of the target animal, and to adjust the control signal transmitted to light source 304 based on the comparison to provide closed-loop insect 310 behavior control.

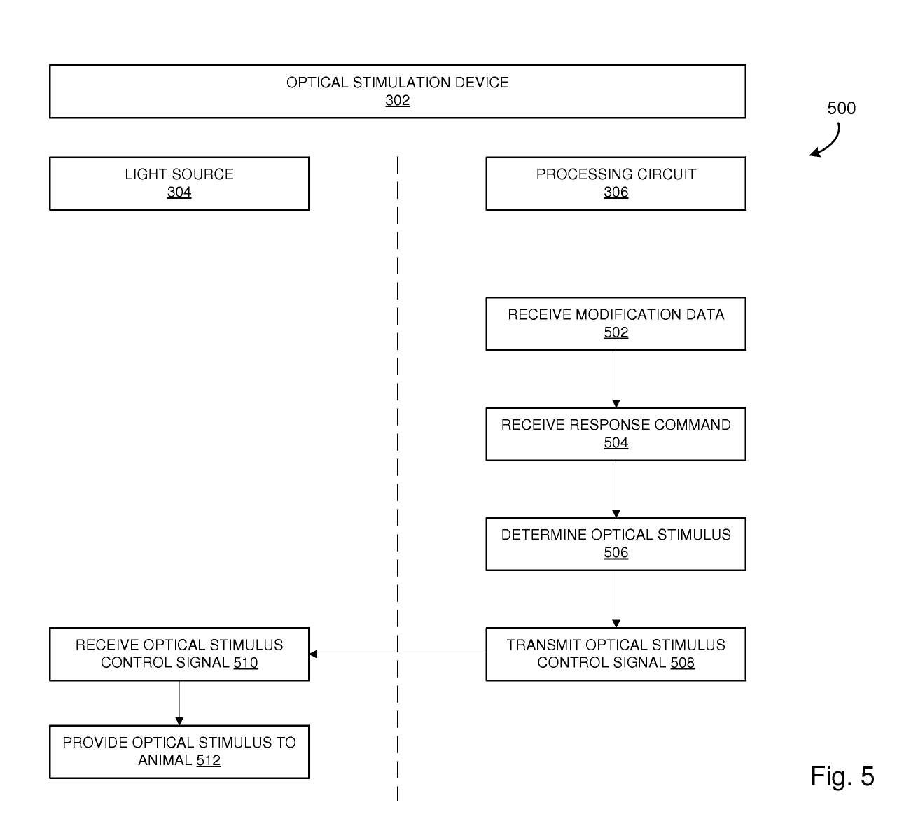

FIG. 5 is a flow diagram of method 500 of operating optical stimulation device 302, including processing circuit 306 and light source 304, as depicted in FIGS. 3 and 4, according to an embodiment. As illustrated in FIG. 4, processing circuit 306 may include, for example, controller 402, including processor 404 and memory 406; user interface module 408; optical stimulation module 314; and behavior analysis module 412.

At 502, modification data is received by processing circuit 306. The modification data may include any information relating to a genetic modification of an animal. For example, the animal may be genetically modified to include nucleic acid encoding a light-responsive element that has been introduced into a target olfactory sensory gene of the animal. The modification data may include, for example, information relating to the particular type (e.g., genus or species) of animal that includes the genetic modification, the particular genetically engineered olfactory sensory protein that has been introduced into the animal, the type of optical stimulus to which the genetically engineered olfactory sensory protein is configured to respond, and/or the particular target olfactory sensory protein(s) into which the light-responsive element has been introduced, etc.

The modification data relating to the light-responsive element that has been introduced into the animal may identify, for example, a gene or nucleic acid sequence of a particular GPCR opsin, such as a rhodopsin, a channelrhodopsin, a halorhodopsin, an archaerhodopsin, a bacteriorhodopsin, etc. The modification data may also identify a particular type of light (e.g., a particular wavelength) to which the genetically engineered olfactory sensory protein, is configured to respond. For example, one genetically engineered olfactory sensory protein may respond to blue light (e.g., 480 nm wavelength) and another genetically engineered olfactory sensory protein may respond to green light (e.g., 532 nm wavelength). In some embodiments, the modification data may identify a plurality of different genetically engineered olfactory sensory proteins that have been introduced into the animal.

The modification data relating to the target olfactory sensory protein into which the nucleic acid encoding a light-responsive element has been introduced may identify a particular OR and/or Orco genes, or combination thereof, into which the opsin-related nucleic acid has been introduced. The particular OR and/or Orco encoded by the particular genetically engineered OR and/or Orco gene, or combination thereof, may define the particular odorant identity that may be perceived by the animal upon the animal being exposed to the optical stimulation.

At 504, a response command regarding a desired behavioral response of the animal is received by processing circuit 306. The desired behavioral response may be locomotion, such as attraction to or repulsion from light source 304. The response command may be received from user interface module 408. For example, a user may provide a response command that includes repelling a particular animal species from light source 304.

At 506, an optical stimulus is determined by processing circuit 306 based on the modification data and the response command. The optical stimulus, when provided to the animal, is configured to cause the animal to exhibit the desired behavioral response. The optical stimulus may include particular properties, such as wavelength and intensity, according to the modification data received at 502.

In some embodiments, the optical stimulus is further based on a measurement signal received from animal position sensor 314. In an embodiment, processing circuit 306 is configured to compare the desired behavioral response of the animal with the actual behavior of the animal, and to adjust the optical stimulus based on the comparison to provide closed-loop behavioral control of the animal.

At 508, a control signal is transmitted by processing circuit 306 to light source 304. The control signal is configured to cause light source 304 to provide the optical stimulus to the animal. In an embodiment, light source 304 is configured to provide the optical stimulus via a light beam (e.g., a laser beam). The light beam may have any of various properties, such as constant and/or time-variable wavelength and intensity, according to the optical stimulus determined at 506.

It is important to note that the construction and arrangement of the elements of the systems and methods as shown in the exemplary embodiments are illustrative only. Although only a few embodiments of the present disclosure have been described in detail, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements. It should be noted that the elements and/or assemblies of the enclosure may be constructed from any of a wide variety of materials that provide sufficient strength or durability, in any of a wide variety of colors, textures, and combinations. Accordingly, all such modifications are intended to be included within the scope of the present inventions. The order or sequence of any process or method steps may be varied or re-sequenced according to alternative embodiments. Other substitutions, modifications, changes, and omissions may be made in the design, operating conditions, and arrangement of the preferred and other exemplary embodiments without departing from scope of the present disclosure or from the spirit of the appended claims.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general purpose computer, special purpose computer, or special purpose processing machines to perform a certain function or group of functions.

Although the figures may show a specific order of method steps, the order of the steps may differ from what is depicted. Also two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method, comprising:
applying, by an optical stimulation device, an optical stimulus to an olfactory organ of an animal having a genetically engineered olfactory sensory protein to produce a desired neural response, wherein the genetically engineered olfactory sensory protein has been genetically modified to incorporate a light-responsive element.

2. The method of claim 1, wherein the light-responsive element is a functional opsin protein.

3. (canceled)

4. The method of claim 1, wherein the desired neural response is configured to cause a desired behavioral response of the animal.

5-6. (canceled)

7. The method of claim 4, further comprising:
monitoring, by a processing circuit of the optical stimulation device, an actual behavioral response of the animal; and
controlling, by the processing circuit, the optical stimulus based on the monitoring.

8. (canceled)

9. The method of claim 1, wherein the olfactory sensory protein comprises an odorant receptor (OR).

10. The method of claim 1, wherein the olfactory sensory protein comprises an odorant co-receptor (Orco).

11. The method of claim 1, wherein the light-responsive element comprises at least a portion of a G-Protein Coupled Receptor (GPCR) opsin.

12-18. (canceled)

19. The method of claim 1, wherein the olfactory sensory protein is a first olfactory sensory protein, the light-responsive element is a first light-responsive element, the desired neural response is a first desired neural response, the optical stimulus is a first optical stimulus, and wherein the first desired neural response is configured to cause a first desired behavioral response of the animal, and further comprising:
applying, by the optical stimulation device, a second optical stimulus to the olfactory organ of the animal to produce a second desired neural response, the olfactory organ further including a second genetically engineered olfactory sensory protein that has been genetically modified to incorporate a second light-responsive element, wherein the second desired behavioral response is different than the first desired response, and wherein the second optical stimulus has at least one of a wavelength and an intensity different than that of the first optical stimulus.

20. The method of claim 19,
wherein the optical stimulation device includes a first light source to produce the first optical stimulus and a second light source to produce the second optical stimulus, and
wherein the first desired behavioral response includes an attraction towards the first light source, and the second desired behavioral response includes a repulsion from the second light source.

21-24. (canceled)

25. The method of claim 1, wherein the insect animal is of the species Anopheles gambiae.

26-55. (canceled)

56. A method, comprising:
receiving, by a processor, optical sensitivity data regarding optical sensitivity of a genetically engineered olfactory sensory protein of an animal;
receiving, by the processor, a response command regarding a desired behavioral response of the animal;
determining, by the processor, based on the optical sensitivity data and the response command, an optical stimulus that, when provided to the animal, causes the animal to exhibit the desired behavioral response; and
transmitting, by the processor, a control signal to the light source to cause the light source to provide the optical stimulus to the animal.

57. The method of claim 56, wherein the genetically engineered olfactory sensory protein is expressed in an olfactory organ of the animal, such that applying the optical stimulus to the olfactory organ causes the genetically engineered olfactory sensory protein to produce a desired neural response, wherein the desired neural response is configured to cause the desired behavioral response.

58. The method of claim 56, wherein the genetically engineered olfactory sensory protein includes an odorant receptor (OR).

59. The method of claim 56, wherein the genetically engineered olfactory sensory protein includes an odorant co-receptor (Orco).

60. The method of claim 56, further comprising monitoring, by the processor, an actual behavior of the animal, wherein the optical stimulus is determined based on the monitored actual behavior.

61. The method of claim 60, further comprising determining, by the processor, the optical stimulus based on a comparison between the desired behavioral response and the monitored actual behavior.

62-68. (canceled)

69. The method of claim 56, wherein the optical sensitivity data relates to the genetically engineered olfactory sensory protein.

70-73. (canceled)

74. The method of claim 56, wherein the animal is of the species Anopheles gambiae.

75-77. (canceled)

78. A method, comprising:
identifying a target olfactory sensory protein of an animal; and
genetically engineering the target olfactory sensory protein to form a genetically engineered olfactory sensory protein incorporating a light-responsive element, wherein the light-responsive element is configured to produce a desired neural response when an optical stimulus is applied to an olfactory organ of the animal, the olfactory organ includes the genetically engineered olfactory sensory protein.

79. The method of claim 78, wherein the light-responsive element is a functional opsin protein.

80. (canceled)

81. The method of claim 78, wherein the desired neural response is configured to cause a desired behavioral response of the animal.

82-84. (canceled)

85. The method of claim 78, wherein the target olfactory sensory protein comprises an odorant receptor (OR).

86. The method of claim 78, wherein the target olfactory sensory protein comprises an odorant co-receptor (Orco).

87. The method of claim 78, wherein the light-responsive element comprises at least a portion of a G-Protein Coupled Receptor (GPCR) opsin.

88-89. (canceled)

90. The method of claim 78, further comprising introducing nucleic acid encoding the genetically engineered olfactory sensory protein into the animal.

91. The method of claim 90, wherein the nucleic acid is introduced into the animal by incorporation in one or more germ-line cellular precursors of the animal.

92. The method of claim 90, wherein the nucleic acid is introduced into the animal by incorporation in one or more ancestors of the animal.

93. The method of claim 90, wherein the nucleic acid is introduced via non-viral transfection.

94. The method of claim 90, wherein the nucleic acid is introduced via viral transfection.

95. The method of claim 94, wherein the viral transfection includes the use of a gene drive.

96. The method of claim 90, wherein the nucleic acid is introduced in a controlled environment.

97. The method of claim 78, further comprising:
selectively breeding the animal with other animals having the genetically engineered olfactory sensory protein, based on a desired trait, to obtain a plurality of offspring having the genetically engineered olfactory sensory protein and the desired trait; and
releasing the plurality of offspring into the wild.

98. The method of claim 78, wherein the genetically engineered olfactory sensory protein is encoded by a first gene, and further comprising introducing a second gene into the animal to provide the animal a genetic enhancement over wild-type animals.

99. The method of claim 98, further comprising, upon introducing the second gene into the animal, selectively breeding the animal with other animals having each of the first gene and the second gene, based on a desired trait, to obtain a plurality of offspring having each of the first gene, the second gene, and the desired trait; and
releasing the plurality of offspring into the wild.

100-102. (canceled)

103. The method of claim 78, wherein the target olfactory sensory protein is a first target olfactory sensory protein, the light-responsive element is a first light-responsive element, the desired neural response is a first desired neural response, the optical stimulus is a first optical stimulus, and wherein the first desired neural response is configured to cause a first desired behavioral response of the animal, and further comprising:
identifying a second target olfactory sensory protein of the animal, the second target olfactory sensory protein different than the first target olfactory sensory protein; and
introducing a second light-responsive element into the second target olfactory sensory protein, wherein the second light-responsive element is configured to produce a second desired neural response in response to a second optical stimulus, wherein the second desired neural response is configured to cause a second desired behavioral response of the animal, the second desired behavioral response being different than the first desired response, the second optical stimulus having at least one of a wavelength and an intensity different than that of the first optical stimulus.

104-114. (canceled)
